(19) United States
(12) Patent Application Publication
(10) Pub. No.: US 2003/0036111 A1
(43) Pub. Date: Feb. 20, 2003

(54) Mitochondrial protein

(76) Inventors: John Walker, Cambridge (GB); Michael Runswick, Cambridge (GB); Ferdinando Palmieri, Bari (IT); Guiseppe Fiermonte, Bari (IT); Vincenza Dolce, Bari (IT)

Correspondence Address: PALMER & DODGE, LLP, KATHLEEN M. WILLIAMS, 111 HUNTINGTON AVENUE, BOSTON, MA, 02199, US

(21) Appl. No.: 09/923,921

(22) Filed: Aug. 7, 2001

(51) Int. Cl. C12Q 1/68; C07H 21/04; C12P 21/02; C12N 5/06; C07K 14/435

(52) U.S. Cl. 435/69.1; 435/320.1; 435/325; 530/350; 536/23.5; 435/6

(57) ABSTRACT

The present application relates to a mitochondrial deoxynucleotide carrier (DNC) which transports deoxynucleoside diphosphates, wherein said carrier: a) catalyses the exchange of dATP for dADP or ADP with first-order kinetics and a rate constant of 0.02 min⁻¹; b) has a pH optimum at pH 6.8; and c) exchanges dATP more efficiently from dNDPs than for NTPs, dNTP, dNMPs and pyrophosphate, as well as to the use of such a carrier in the design of nucleoside analogue drugs.

The present invention relates to a mitochondrial deoxynucleotide carrier (DNC). In particular, the invention relates to a polypeptide related to mammalian adenine nucleotide carriers (ANCs), which is responsible for deoxynucleotide diphosphate transport into mitochondria, and to methods of modulating the toxic effects of chemotherapeutic and antiviral compounds.

Nucleoside analogues were among the first compounds shown to be effective against viral infections. Acyclovir, the first of such drugs, is used extensively in the treatment of herpetic infections. The first four anti-HIV drugs to be approved, AZT, ddI, ddC and D4T, were also nucleoside analogues. All four of these drugs and other nucleoside analogues are believed to have a similar mechanism of viral inhibition, in which the nucleosides are progressively phosphorylated to a 5′-triphosphate, which then acts as a chain terminator in the viral reverse transcriptase (RT) reaction. They are thus often referred to as nucleoside reverse transcriptase inhibitors (NRTIs). Anti-viral activity is dependent on the intracellular phosphorylation of the analogue and the ability of the phosphorylated analogue to interact with the viral RT. The rate limiting step in most cells is believed to be the initial phosphorylation by nucleoside kinases, or in the case of AZT, the conversion of a nucleoside monophosphate to a nucleoside diphosphate.

The major limitation of nucleoside analogues is their toxicity. Toxic side effects vary from compound to compound: anaemia and/or neutropenia are frequently seen with AZT; neutropenia and peripheral neuropathy with 3TC; peripheral neuropathy with ddC , D4T and ddI; and acute pancreatitis with ddI.

NRTIs generally do not affect DNA synthesis in the nuclei of somatic cells because the cellular DNA polymerases possess a “proof-reading” mechanism that removes the nucleoside analogues if they are inserted into a new DNA chain. Mitochondrial genes, however, are particularly prone to damage. DNA polymerase γ, which directs replication of mitochondrial genes, differs the DNA polymerase enzymes found in the cell nucleus. Polymerase γ has no “proof-reading” function, so little repair of errors occurs when during DNA synthesis. Nucleoside analogues therefore inhibit DNA polymerase γ in the same way they inhibit reverse transcriptase. Tumour cells, moreover, may have deficient proof-reading mechanisms; nucleoside drugs are thus effective against tumours, as their toxicity to tumour cells is greater than that to normal cells.

Mitochondria rely on genetic redundancy to protect against errors in their genes. Defective DNA coexists and replicates alongside correct DNA, which covers for the defect. Similarly, functional mitochondria can compensate for defective mitochondria within the same cell. The system breaks down when damaged mitochondrial DNA reaches a threshold proportion above 70%. Cells then begin to suffer from energy deficiencies and turn increasingly to anaerobic processes. Anaerobic respiration is much less efficient than oxidative phosphorylation.

Specific disease syndromes are also connected with rare inherited mitochondrial mutations. Mitochondria-related diseases vary in severity from person to person, and symptoms frequently appear only as a person ages. Tissues such as muscles and nerves, which require high levels of energy, are most often involved. Some of the specific conditions related to inadequately low mitochondrial activity are muscle wasting (myopathy); heart failure (cardiomyopathy); peripheral numbness and pain (neuropathy); generalised loss of the kidney's ability to filter the blood (proximal renal tubular dysfunction or Fanconi-like syndrome); low blood cell counts (anaemia, leukopenia, thrombocytopenia, or pancytopenia); swelling and fatty degeneration of the liver (hepatomegaly with steatosis); and pancreatic inflammation (pancreatitis). Fatigue, psychological depression, and high lactic acid levels (lactic acidosis) are more generalised signs.

Nucleoside entry and exit from mammalian cells is mediated by nucleoside-specific membrane transporters. Nucleoside transport processes comprise a diverse array that includes facilitated diffusion processes as well as concentrative, sodium-dependent, secondary active transport processes.

The inner membranes of mitochondria contain a family of proteins that transport various substrates and products into and out of the matrix. Family members have three tandem-repeated sequences, each of about 100 amino acids, made of two hydrophobic transmembrane a-helices joined by a large hydrophilic segment (thought to be an extramembranous loop; refs. 1-3). The tandem repeats contain conserved features. So far, 11 members of the family have been identified and sequenced. They are the uncoupling protein, and carriers for adenine nucleotides (ANC), phosphate, oxoglutarate, citrate, dicarboxylates, carnitine, ornithine, succinate-fumarate, oxaloacetate-sulfate, and oxodicarboxylates (1-7). The functions of other family members found in genome sequences are unknown.

### SUMMARY OF THE INVENTION

We have isolated and cloned a novel mitochondrial carrier polypeptide.

In accordance with a first aspect of the present invention, there is provided a mitochondrial deoxynucleotide carrier (DNC) which transports transport deoxynucleoside diphosphates, wherein said carrier:

a) catalyses the exchange of dATP for dADP or ADP with first-order kinetics and a rate constant of 0.02 min⁻¹;

b) has a pH optimum at pH6.8; and

c) exchanges dATP more efficiently from dNDPs than for NTPs, dNTP, dNMPs and pyrophosphate.

The protein according to the invention has been overexpressed in bacteria, purified, and reconstituted into phospholipid vesicles, where it is found to transport deoxynucleoside diphosphates (or, albeit less efficiently, deoxynucleoside triphosphates,). The function of the protein is to act as a deoxynucleotide carrier (DNC) to supply precursors of mitochondrial DNA synthesis in the mitochondrial matrix. The transport occurs in exchange for dNDPs, ADP or ATP. Thus, whilst the polypeptide is characterised having regard to exchange of ATP, it will be understood that alternative exchange nucleotides may be used in the characterisation procedures.

Preferably, the protein according to the invention has a calculated molecular mass of 34,588.

According to a preferred aspect of the present invention, there is provided a mitochondrial deoxynucleotide carrier (DNC) which transports transport deoxynucleoside diphosphates, wherein said carrier:

a) has the amino acid sequence set forth in SEQ. ID. No. 2; or

b) has an amino acid sequence as set forth in SEQ. ID. No. 2, including one or more amino acid additions, deletions or substitutions, and retains the ability to transport deoxynucleoside diphosphates; or

c) is encoded by a nucleic acid sequence set forth in SEQ. ID. No. 1.

Preferably, the protein according to the invention is a mammalian protein, more preferably a primate protein and advantageously a human protein.

The protein also provides a route for uptake into the organelle of toxic nucleoside analogues, such as 3′-azido-3′-deoxythymidine. Thus, the invention provides a method for selecting a nucleoside analogue, comprising assaying the efficiency with which the nucleoside analogue is transported in exchange for ADP by a protein according to the invention; and selecting those analogues which are least effectively transported.

Nucleoside analogues which are poor substrates for a DNC according to the invention are likely to show reduced mitochondrial toxicity when used as NRTIs in antiviral or antitumour therapy. Accordingly, screening may be used either to eliminate candidate drugs which are effectively transported into the mitochondrion before any further testing is carried out, or to screen candidate drugs which has already been shown to have some antiviral or antitumour activity to identify those which will possess the lowest toxicity.

In a further aspect of the present invention, therefore, there is provided the use of a DNC according to the invention in the screening of nucleoside analogues for toxic side-effects.

Moreover, there is provided a method for identifying a compound or compounds capable, directly or indirectly, of modulating the uptake of nucleoside analogues by a DNC according to the invention, and thereby its the toxicity of said nucleoside analogues, comprising the steps of:

(a) incubating a DNC according to the invention with the compound or compounds to be assessed; and

(b) identifying those compounds which influence the activity of the DNC.

Advantageously, the DNC is incubated in membrane-bound form, such as part of a reconstituted phospholipid vesicle system or other transport modelling system, together with the nucleoside analogue and ADP, ATP or a dNDP. Transport of the nucleoside analogue across a membrane may be measured using known membrane transport assays, for example as described below.

Furthermore, the invention provides methods for producing polypeptides capable of modulating DNC activity, including expressing nucleic acid sequences encoding them, methods of modulating DNC activity in cells in vivo, and methods of treating mitochondrial diseases involving nucleoside transport.

In a still further aspect, the invention provides nucleic acids encoding the DNC according to the invention. Advantageously, the nucleic acid comprises a nucleotide sequence selected from the group consisting of: the nucleotide sequence of: (a) SEQ. ID. No. 1; (b) the coding portion of the nucleotide sequence of SEQ. ID. No. 1; (c) a nucleotide sequence which is at least 80% homologous to (a) or (b); and (d) a nucleotide sequence at least 20 nucleotides in length which hybridises under stringent conditions with (a), (b) or (c).

The DNC according to the invention is referred to below simply as “DNC”, which term encompasses the mitochondrial DNC molecule(s) as described and claimed herein.

### BRIEF DESCRIPTION OF THE FIGURES

FIG. 1. Sequence of a human cDNA and the encoded DNC. Amino acids are numbered from 1-320. An asterisk denotes the stop codon. Primers and probes are shaded. The nested primers 1F/2F and 1R/2R and probe 1P were used to confirm the EST sequence. The partial cDNA sequence was extended in 3′ and 5′ directions with primers AP1 and AP2 and nested oligonucleotides 3F/4F or 3R/4R, respectively. Primers RT 1F and RT 1R and probe RT 1P were used in reverse transcription-PCR experiments. Horizontal arrows pointing right and left indicate that primers were synthesised as shown or as the complement, respectively.

FIG. 2. Purification of DNC by Ni⁺-agarose affinity chromatography. Proteins were separated by SDS/PAGE and stained with Coomassie blue. Lane M, markers (BSA, carbonic anhydrase, and cytochrome c); lane 1, sarkosyl extract of inclusion bodies; lane 2, pH 6.8 eluate; lane 3, pH 6.5 eluate; lane 4, purified DNC, eluted at pH 6.2. The position of DNC is indicated on the right by an arrow.

FIG. 3. Time course of dATP/ADP exchange and substrate specificity of human DNC. (a) Time course of [α-35 S]dATP/ADP exchange in proteoliposomes reconstituted with the recombinant DNC. [α-35 S]dATP (1 mM) was added to proteoliposomes containing 10 mM ADP (⋄) or 10 mM NaCl (∇). (b) Dependence of DNC activity on internal substrate. Proteoliposomes were preloaded internally with various substrates (concentration 10 mM). Trans-port was started by addition of 20 mM [α-35 S]dATP and stopped after 2 min. The values are means 6 SD of at least three experiments. (c) Inhibition of the rate of [α-35 S]dATP uptake by external substrates. Proteoliposomes were preloaded internally with 10 mM ADP. Transport was started by adding 125 mM [α-35 S]dATP and stopped after 2 min. External substrates (concentration 0.5 mM) were added together with [α-35 S]dATP. The extents of inhibition (%) from a representative experiment are reported. The control value for uninhibited exchange was 0.45 mmol/min per gram of protein.

FIG. 4. Expression of human DNC in various tissues. Analysis of total RNA from human (h) and mouse (m) tissues (A). (a) Hybridisation of cDNA fragments for the DNC with probe RT 1P. (b) Ethidium-bromide-stained cDNA fragments for b-actin. (B) Immunodetection of the DNC in mitochondria isolated from rat tissues. In a and b, mitochondria (150 mg of protein) and human DNC (75 ng) were exposed to antisera to the DNC and subunit IV of the cytochrome c oxidase, respectively.

FIG. 5. Folding of the DNC in the inner membranes of mitochondria. The topography of the six transmembrane α-helices is based on the hydrophobic profile of the sequence in FIG. 1. Each of the three tandem repeats in the sequence is folded into two transmembrane α-helices with a large intervening hydrophilic loop. The three repetitive elements are linked by shorter loops. The cytoplasmic and matrix locations of the various features are based on experimental evidence of locations of analogous features in other members of the family of mitochondrial carriers. The sequences in black are related to the DNA-binding domain of the nuclear receptor family. Residues 241-243 (in squares) correspond to the sequence RRR at residues 234-236 of the ANC from Saccharomyces cerevisiae.

### DETAILED DESCRIPTION OF THE INVENTION

Although in general the techniques mentioned herein are well known in the art, reference may be made in particular to Sambrook et al., Molecular Cloning, A Laboratory Manual (1989) and Ausubel et al., Short Protocols in Molecular Biology (1999) 4th Ed, John Wiley & Sons, Inc (as well as the complete version Current Protocols in Molecular Biology.

1. Nucleoside Analogues

Nucleoside analogue drugs are well known in the art and used to treat viral infection, particularly retroviral infection, as well as tumours. As used herein, the term “nucleoside analogues” refers to any derivative of a natural nucleoside which is capable of being incorporated into a nucleic acid by mitochondrial polymerase γ, but which when incorporated into the nucleic acid prevents the functional synthesis or operation of that nucleic acid molecule.

For example, any nucleoside dideoxy (dd) derivative is a nucleoside analogue according to the present invention, because incorporation of a ddNTP leads to chain synthesis termination.

Currently, in the UK, the following nucleoside analogues are licensed for medical use: zidovudine (AZT, Retrovir); 3TC (lamivudine, Epivir); Combivir (AZT+3TC fixed dose formulation); ddI (didanosine, Videx); ddC (zalcitabine, HIVID); d4T (stavudine, Zerit); and Abacavir (1592U89, Ziagen).

Other known nucleoside analogues include 5-substituted derivatives of conformationally locked nucleoside analogues, which are useful as antiviral and anticancer agents. The 5-substituent may be a halogen, alkyl, alkene, halovinyl or alkyne group, and the nucleotide base may be cytosine or uracil; 2′,3′-dideoxy-3′-C-(hydroxymethyl)-4′-thionucleosides; 1,3-dioxolan-, 1,3-oxathiolan-, and 1,3-dithiolan-2-ylnucleosides; Acyclovir; AzdU, BCH-10652; BW 935U83; FTC; Lodenosine; PMEA; Ribavirin; and Trizivir.

The present invention provides the methodology for the person skilled in the art to test such nucleoside analogues, other known nucleoside analogues and nucleoside analogues which will be developed in the future for mitochondrial toxicity in a simple in vitro assay.

2. Transport Assay Systems

Many systems are available in the art for assessing transport across biological membranes. For example, systems are available which employ membrane vesicles containing the desired protein, in this case DNC. The performance of the protein in transporting nucleosides across the membrane may be assessed, for example, by measuring the rate of incorporation of radioactive nucleosides into the vesicles in exchange for material present in the vesicle lumen. For example, see Baldwin, S (2000) Membrane Transport: A Practical Approach. ISBN 0-19-963705-9; Oxford University Press, UK.

An exemplary transport assay system, using reconstituted liposome vesicles, is described in detail below.

3. Deoxynucleotide Carriers (DNC)

The invention provides a novel deoxynucleotide carrier which has the amino acid sequence set forth in SEQ. ID. No. 2. The sequence reported herein has been deposited in the EMBL database (accession no. AJ25 1857).

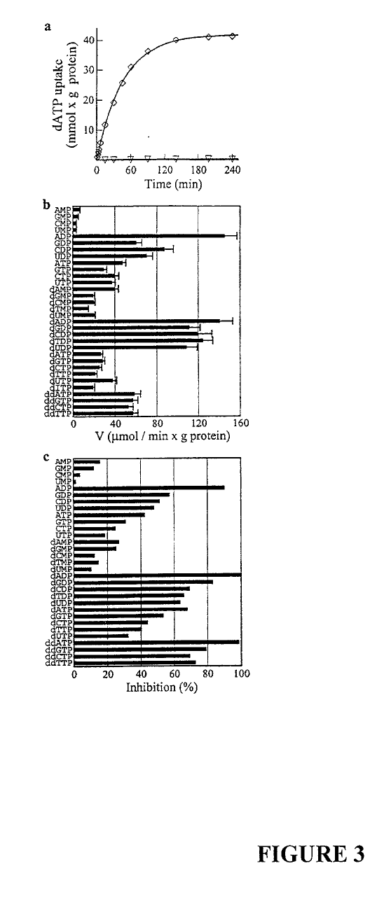

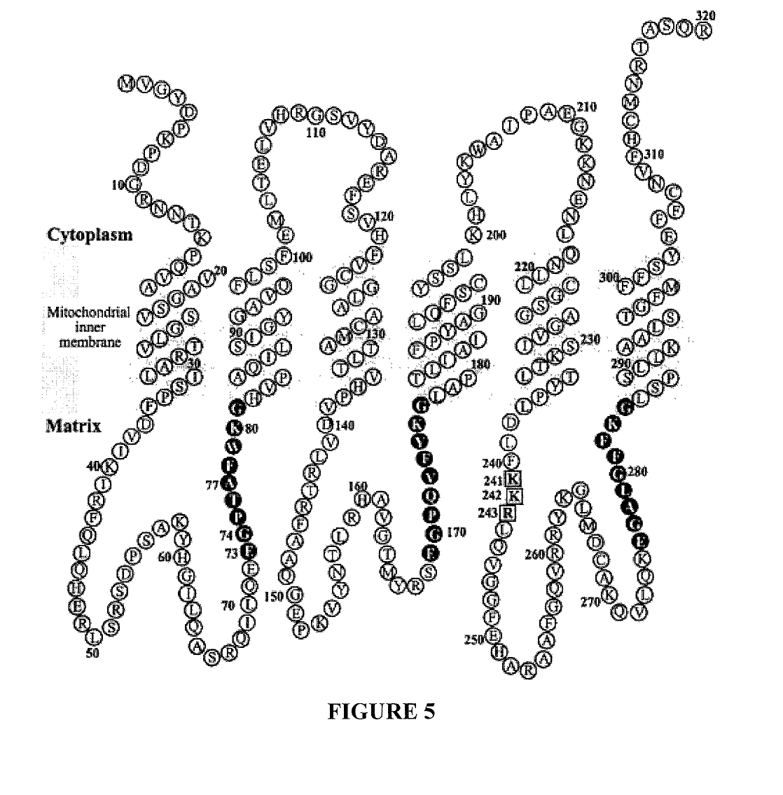

The sequence of the human DNC consists of three homologous repeats of about 100 amino acids and contains sequence motifs characteristic of the mitochondrial carrier family. Like other family members, it appears to have six transmembrane α-helices (FIG. 5). It is about 22% identical to mammalian ANCs. The ANCs contain a strictly conserved motif (RRR) in the third large hydrophilic loop thought to be involved in nucleotide binding (12) and a similar motif (KKR) is at residues 241-243 of the DNC, presumably fulfilling a similar role. The DNC also contains a sequence at residues 73-77 in the first large loop that conforms to the sequence motif EGXXA, the P-box of the DNA-binding domain of nuclear receptors (13). The same motif is also found in the loop connecting the fifth and sixth a-helices in mammalian ANCs and in the uncoupling protein (UCP1) (12, 14). In rat UCP1, the motif is thought to be involved in controlling its activity via GDP binding. Therefore, it is likely that the P-box motif in the DNC is also involved in binding nucleotide substrates or, alternatively, that it interacts directly with mtDNA. Reconstituted DNC catalyses an exchange reaction between nucleotides and deoxynucleotides. The best internal substrates are dNDPs and ADP, whereas dNDPs, dNTPs, and NDPs are the best external ones (dNDPs have the highest affinity). If the carrier is oriented in the liposomal membrane as in mitochondria, it is likely that the DNC catalyses the uptake of dNDPs into the mitochondrial matrix. All dNDPs are transported by DNC. Once in the mitochondrion, the dNDPs will be converted to the corresponding triphosphate and incorporated into the mtDNA by the DNA polymerase-γ. Because ribonucleotide reductase is found in the cytosol of eukaryotic cells (15), the DNC appears to be essential for mtDNA synthesis. The higher Ki values for the dUDP and dUTP (Table 1), which are not incorporated into DNA, support the view that DNC is involved primarily in the mtDNA synthesis. Radioactive dNTPs are taken up by isolated mitochondria and incorporated into mtDNA (16-18) but, because they have lower affinities for the DNC than dNDPs, it is unlikely that they are its physiological substrates. The internal counterion for exchange could be ADP or ATP (FIG. 3b), but ATP is exchanged at a lower rate. In the resting state, the intramitochondrial ATP/ADP ratio is about 4 (19), and the rate of exchange of external dNDPs for internal ATP would be favoured by the proton electrochemical gradient generated by electron transport. Internal GDP was exported rather poorly (FIG. 3b) and, in comparison with adenine nucleotides, is present in the mitochondrial matrix in minute amounts. It is improbable that internal GDP is the physiological counteranion for the uptake of dNDPs.

Human DNCs can exchange ddNTPs much more efficiently than the corresponding deoxy analogues (FIG. 3b and c). Furthermore, the inhibition constants of external ddNTPs are close to those of dNDPs (Table 1). Therefore, ddNDPs (which are not available commercially) may well be the best substrates to be transported by the DNC. These properties suggest that the DNC is involved directly in the cytotoxicity of antiviral and anticancer nucleoside analogues such as 2′,3′-dideoxycytidine, 2′,3′-dideoxyinosine, and 3′-azido-3′-deoxythymidine. Cytoplasmic kinases convert these and other dideoxynucleosides to their mono-, di-, and triphosphate derivatives (17, 20). The latter two products would be expected to be transported into mitochondria by the DNC, there to inhibit the synthesis of mtDNA by competing with dNTPs for the active site of the DNA polymerase-γ and by chain termination (21). Clinical and laboratory findings have shown that the mechanism of toxicity of most antiviral and anticancer nucleoside analogues is to impair mitochondrial function (17, 20, 22-25). In fact, the main side effects of these drugs, myopathy, cardiomiopathy, polyneuropathy, and lactic acidosis, greatly resemble the spectrum of clinical manifestations seen in inherited mitochondrial diseases (26). Furthermore, after prolonged assumption of these drugs, histological findings commonly associated with depletion of mtDNA, such as red-ragged fibres, are observed (27). It should be noted that the antiviral nucleotide analogues strongly interfere with the action of the viral reverse transcriptases and the mtDNA polymerase-γ but have a very low affinity for the nuclear DNA polymerases (17, 28, 29).

The invention moreover relates to variants of the sequence set forth herein. It will be understood that DNC polypeptide sequences for use in the methods of the invention are not limited to the particular amino acid sequences shown in SEQ ID No. 2 or fragments thereof but also include homologous sequences obtained from any source, typically mitochondria derived from other mammalian species.

Thus, the present invention encompasses the use of variants, homologues or derivatives of the amino acid sequences of SEQ ID No. 2, as well as variants, homologues or derivatives of the amino acid sequences coded for by the nucleotide sequence shown in SEQ ID No. 1.

In the context of the present invention, a homologous sequence is taken to include an amino acid sequence which is at least 50, 60, 70, 80 or 90% identical, preferably at least 95 or 98% identical at the amino acid level over at least 50, 100 or 200 amino acids with the amino acid sequences of SEQ ID No. 2. In particular, homology should typically be considered with respect to those regions of the sequence essential for oxodicarboxylate transport rather than non-essential neighbouring regions. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate percentage (%) homology between two or more sequences.

Percentage homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids). However, most sequence comparison methods are designed to produce optimal alignments that take into consideration possible insertions and deletions without penalising unduly the overall homology score. This is achieved by inserting “gaps” in the sequence alignment to try to maximise local homology. Common algorithms used to carry out sequence comparisons and calculate homology are implemented in software such as the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program, typically with the default matrix and gap penalties.

Homologous polypeptides may be obtained, for example by cloning the corresponding nucleotides sequences using a variety of well-known techniques. For example, probes comprising all or part of SEQ ID. No. 1 may be used to probe DNA libraries made from other mitochondria under conditions of medium to high stringency. Such techniques may also be used to obtain allelic variants.

Variants and strain/species homologues may also be obtained using degenerate PCR which will use primers designed to target sequences within the variants and homologues, often encoding conserved amino acid sequences within the DNC sequence provided herein. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences. It will be appreciated by the skilled person that overall nucleotide homology between sequences from distantly related organisms is likely to be very low and thus in these situations degenerate PCR may be the method of choice rather than screening libraries with labelled fragments of SEQ ID. No. 1

Alternatively, such polynucleotides may be obtained by site directed mutagenesis of characterised sequences, such as SEQ ID. Nos 1 and 2. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

The terms “variant” or “derivative” in relation to the DNC amino acid sequences for use in the present invention includes any substitution of, variation of, modification of, replacement of, deletion of or addition of one (or more) amino acids from or to the sequence providing the resultant amino acid sequence preferably has the ability to transport deoxynucleotides, preferably having at least 25 to 50% of the activity as the polypeptide presented in the sequence listings, more preferably at least substantially the same activity. This may be tested, for example, by reconstituting recombinantly produced proteins into liposomes and determining transport of labelled nucleotides as described in the examples.

Thus DNC sequences may be modified for use in the present invention. Typically, modifications are made that maintain the transport activity of the polypeptide. Thus, in one embodiment, amino acid substitutions may be made, for example from 1, 2 or 3 to 10, 20 or 30 substitutions provided that the modified sequence retains at least about 25 to 50% of, or substantially the same transport activity as the sequences shown herein. As mentioned above, this may be tested, for example, by reconstituting recombinantly produced proteins into liposomes and determining transport of labelled nucleotides as described in the examples.

However, in an alternative embodiment, modifications to the amino acid sequences of a DNC polypeptide may be made intentionally to reduce the biological activity of the polypeptide. For example truncated polypeptides that transport natural nucleotides but fail to transport toxic nucleoside analogues across the mitochondrial membrane may be useful as inhibitors of the biological activity of the natural molecule and may function to reduce the toxicity of nucleoside analogue drugs.

In general, preferably less than 20%, 10% or 5% of the amino acid residues of a variant or derivative are altered as compared with the corresponding region depicted in the sequence listings.

Conservative substitutions may be made, for example according to the Table below. Amino acids in the same block in the second column and preferably in the same line in the third column may be substituted for each other:

| | | |
|---|---|---|
| ALIPHATIC | Non-polar | GAP |
| | | ILV |
| | Polar-uncharged | CSTM |
| | | NQ |
| | Polar-charged | DE |
| | | KR |
| AROMATIC | | HFWY |

Polypeptides of the invention also include fragments of the above mentioned fall length polypeptides and variants thereof, including fragments of the sequences set out herein. Suitable fragments will typically be at least about 50, 100, 150 or 200 amino acids in length and retain the ability to transport deoxynucleotides across the mitochondrial membrane. Polypeptide fragments of the DNC proteins and allelic and species variants thereof may contain one or more (e.g. 2, 3, 5, or 10) substitutions, deletions or insertions, including conserved substitutions. Where substitutions, deletion and/or insertions have been made, for example by means of recombinant technology, preferably less than 20%, 10% or 5% of the amino acid residues depicted in the sequence listings are altered.

The DNC proteins for use in the present invention are typically made in vivo by recombinant means as described below. Since DNC proteins have been shown herein to be located in the mitochondrial membrane, generally, DNC proteins and nucleotides encoding the same will contain targeting sequences to ensure that the proteins are expressed and targeted to the correct location in the mitochondrial membrane. The native DNC mitochondrial signal sequences may be used. Alternatively, other suitable mitochondrial signal sequences may be used.

Polynucleotides for use in the invention comprise nucleic acid sequences encoding DNC amino acid sequences as described above. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

DNC polynucleotides for use in the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of the polynucleotides.

Given the guidance provided herein, the nucleic acids of the invention are obtainable according to methods well known in the art. For example, a DNA of the invention is obtainable by chemical synthesis, using polymerase chain reaction (PCR) or by screening a genomic library or a suitable cDNA library prepared from a source believed to possess a mitochondrial DNC and to express it at a detectable level.

Chemical methods for synthesis of a nucleic acid of interest are known in the art and include triester, phosphite, phosphoramidite and H-phosphonate methods, PCR and other autoprimer methods as well as oligonucleotide synthesis on solid supports. These methods may be used if the entire nucleic acid sequence of the nucleic acid is known, or the sequence of the nucleic acid complementary to the coding strand is available. Alternatively, if the target amino acid sequence is known, one may infer potential nucleic acid sequences using known and preferred coding residues for each amino acid residue.

An alternative means to isolate the gene encoding a DNC is to use PCR technology as described e.g. in section 14 of Sambrook et al., 1989. This method requires the use of oligonucleotide probes that will hybridise to DNC nucleic acid. Strategies for selection of oligonucleotides are described below.

Libraries are screened with probes or analytical tools designed to identify the gene of interest or the protein encoded by it. For cDNA expression libraries suitable means include monoclonal or polyclonal antibodies that recognise and specifically bind to DNC; oligonucleotides of about 20 to 80 bases in length that encode known or suspected DNC cDNA from the same or different mitochondrial species; and/or complementary or homologous cDNAs or fragments thereof that encode the same or a hybridising gene. Appropriate probes for screening genomic DNA libraries include, but are not limited to oligonucleotides, cDNAs or fragments thereof that encode the same or hybridising DNA; and/or homologous genomic DNAs or fragments thereof.

A nucleic acid encoding DNC may be isolated by screening suitable cDNA or genomic libraries under suitable hybridisation conditions with a probe, i.e. a nucleic acid disclosed herein. Suitable libraries are commercially available or can be prepared e.g. from cell lines, tissue samples, and the like.

As used herein, a probe is e.g. a single-stranded DNA or RNA that has a sequence of nucleotides that includes between 10 and 50, preferably between 15 and 30 and most preferably at least about 20 contiguous bases that are the same as (or the complement of) an equivalent or greater number of contiguous bases set forth herein. The nucleic acid sequences selected as probes should be of sufficient length and sufficiently unambiguous so that false positive results are minimised. The nucleotide sequences are usually based on conserved or highly homologous nucleotide sequences or regions of DNC. The nucleic acids used as probes may be degenerate at one or more positions. The use of degenerate oligonucleotides may be of particular importance where a library is screened from a species in which preferential codon usage in that species is not known.

Preferred regions from which to construct probes include 5′ and/or 3′ coding sequences, sequences predicted to encode ligand binding sites, and the like. For example, either the full-length cDNA clone disclosed herein or fragments thereof can be used as probes. Preferably, nucleic acid probes of the invention are labelled with suitable label means for ready detection upon hybridisation. For example, a suitable label means is a radiolabel. The preferred method of labelling a DNA fragment is by incorporating α³²P dATP with the Klenow fragment of DNA polymerase in a random priming reaction, as is well known in the art. Oligonucleotides are usually end-labelled with γ³²P-labelled ATP and polynucleotide kinase. However, other methods (e.g. non-radioactive) may also be used to label the fragment or oligonucleotide, including e.g. enzyme labelling, fluorescent labelling with suitable fluorophores and biotinylation.

After screening the library, e.g. with a portion of DNA including substantially the entire DNC-encoding sequence or a suitable oligonucleotide based on a portion of said DNA, positive clones are identified by detecting a hybridisation signal; the identified clones are characterised by restriction enzyme mapping and/or DNA sequence analysis, and then examined, e.g. by comparison with the sequences set forth herein, to ascertain whether they include DNA encoding a complete DNC (i.e., if they include translation initiation and termination codons). If the selected clones are incomplete, they may be used to rescreen the same or a different library to obtain overlapping clones. If the library is genomic, then the overlapping clones may include exons and introns. If the library is a cDNA library, then the overlapping clones will include an open reading frame. In both instances, complete clones may be identified by comparison with the DNAs and deduced amino acid sequences provided herein.

The terms “variant”, “homologue” or “derivative” in relation to the DNC nucleotide sequences for use in the present invention include any substitution of, variation of, modification of, replacement of, deletion of or addition of one (or more) nucleic acid from or to the sequence providing the resultant nucleotide sequence codes for a polypeptide having DNC transport activity, preferably having at least the same activity as the polypeptide sequence presented in the sequence listings.

As indicated above, with respect to sequence homology, preferably there is at least 75%, more preferably at least 85%, more preferably at least 90% homology to the sequences shown in the sequence listing herein. More preferably there is at least 95%, more preferably at least 98%, homology. Nucleotide homology comparisons may be conducted as described above. A preferred sequence comparison program is the GCG Wisconsin Bestfit program described above, using the default parameters.

Also suitable for use in the present invention are nucleotide sequences that are capable of hybridising selectively to the sequences presented herein, or any variant, fragment or derivative thereof, or to the complement of any of the above. Nucleotide sequences are preferably at least 300 nucleotides in length, more preferably at least 450, 600 or 750 nucleotides in length.

The term “selectively hybridisable” means that the polynucleotide used as a probe based on the nucleotides sequences shown in the sequence listings is used under conditions where a target DNC polynucleotide is found to hybridise to the probe at a level significantly above background conditions (e.g. 65° C. and 0.1×SSC {1×SSC=0.15 M NaCl, 0.015 M Na₃ Citrate pH 7.0}). The background hybridisation may occur because of other polynucleotides present, for example, in the cDNA or genomic DNA library being screening. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

4. DNC is a Drug Development Target

DNC according to the present invention has utility as a screening tool, useful for identifying nucleoside analogues with reduced (or increased) mitochondrial toxicity as described hereinbefore. Thus, the invention provides a method for selecting a nucleoside analogue, comprising assaying the efficiency with which the nucleoside analogue is transported in exchange for ADP by a protein according to the invention; and selecting those analogues which are least effectively transported. Transport assays as described above may be used to determine nucleoside analogue transport.

Advantageously, a reference transport efficiency is established in respect of nucleoside analogues currently having pharmaceutical application; novel nucleoside analogues may be compared to said reference efficiency. Nucleoside analogues which are transported more efficiently by DNC are more toxic in vivo; those transported less efficiently are less toxic.

According to a further aspect of the present invention, a DNC molecule is used as a target to identify compounds, for example lead compounds for pharmaceuticals, which are capable of modulating the activity of DNC to reduce or prevent the uptake of nucleoside analogues into mitochondria. Accordingly, the invention relates to an assay and provides a method for identifying a compound or compounds capable, directly or indirectly, of modulating the activity of DNC, comprising the steps of:

(a) incubating DNC with the compound or compounds to be assessed; and

(b) identifying those compounds which influence the activity of DNC.

4a. DNC Binding Compounds

According to a first embodiment of this aspect invention, the assay is configured to detect polypeptides which bind directly to DNC.

The invention therefore provides a method for identifying a modulator nucleoside analogue-induced mitochondrial toxicity, comprising the steps of:

(a) incubating a DNC molecule with the compound or compounds to be assessed; and

(b) identifying those compounds which bind to the DNC molecule.

Preferably, the method further comprises the step of:

(c) assessing the compounds which bind to DNC for the ability to modulate DNC activity in a transport assay.

Binding to DNC may be assessed by any technique known to those skilled in the art. Examples of suitable assays include the two hybrid assay system, which measures interactions in vivo, affinity chromatography assays, for example involving binding to polypeptides immobilised on a column, fluorescence assays in which binding of the compound(s) and DNC is associated with a change in fluorescence of one or both partners in a binding pair, and the like. Preferred are assays performed in vivo in cells, such as the two-hybrid assay.

4b. Compounds Which Modulate DNC Activity

As used herein, “DNC activity” may refer to any activity of DNC, but in particular refers to the nucleoside analogue transporting activity of DNC. Accordingly, the invention may be configured to detect the transport of nucleoside compounds by DNC, and the modulation of this activity by potential therapeutic agents.

Examples of compounds which modulate the transport activity of DNC include dominant negative mutants of DNC itself. Such compounds are able to compete for the nucleosides, thus reducing the activity of DNC in a biological or artificial system. Thus, the invention moreover relates to compounds capable of modulating the nucleoside transport activity of DNC.

In a preferred aspect of this embodiment, the invention provides a method for identifying a lead compound for a pharmaceutical useful in the alleviation of nucleoside analogue toxicity, comprising incubating a compound or compounds to be tested with a DNC molecule, under conditions in which, but for the presence of the compound or compounds to be tested, DNC transports nucleoside analogues in a transport assay with a reference efficiency;

determining the transport efficiency of DNC in the presence of the compound or compounds to be tested; and

selecting those compounds which modulate the transport efficiency of DNC with respect to the reference transport efficiency.

As used herein, “efficiency” refers to the rate at which transport occurs or to the total amount of nucleoside analogue which is transported. Advantageously, it is the rate of nucleoside analogue transport, and may be measured in terms of the rate constant. The reference rate constant is preferably about 0.02 min⁻¹, although it will be understood that this will vary according to the assay conditions which are used.

Preferably, therefore, the assay according to the invention is calibrated in absence of the compound or compounds to be tested, or in the presence of a reference compound whose activity in the presence of DNC is known or is otherwise desirable as a reference value. For example, in a two-hybrid system, a reference value may be obtained in the absence of any compound. Addition of a compound or compounds which increase the transport efficiency of DNC increases the readout from the assay above the reference level, whilst addition of a compound or compounds which decrease this efficiency results in a decrease of the assay readout below the reference level.

5. Compounds

In a still further aspect, the invention relates to a compound or compounds identifiable by an assay method as defined in the previous aspect of the invention. Accordingly, there is provided the use of a compound identifiable by an assay as described herein, for the modulation of nucleoside analogue toxicity.

Compounds which influence the activity of DNC may be of almost any general description, including low molecular weight compounds, including organic compounds which may be linear, cyclic, polycyclic or a combination thereof, peptides, polypeptides including antibodies, or proteins. In general, as used herein, “peptides”, “polypeptides” and “proteins” are considered equivalent.

5a. Antibodies

Antibodies, as used herein, refers to complete antibodies or antibody fragments capable of binding to a selected target, and including Fv, ScFv, Fab′ and F(ab′)₂, monoclonal and polyclonal antibodies, engineered antibodies including chimeric, CDR-grafted and humanised antibodies, and artificially selected antibodies produced using phage display or alternative techniques. Small fragments, such Fv and ScFv, possess advantageous properties for diagnostic and therapeutic applications on account of their small size and consequent superior tissue distribution.

The antibodies according to the invention are especially indicated for therapeutic applications. Accordingly, they may be altered antibodies comprising an effector protein such as a toxin or a label. Especially preferred are labels which allow the imaging of the distribution of the antibody in vivo. Such labels may be radioactive labels or radioopaque labels, such as metal particles, which are readily visualisable within the body of a patient. Moreover, the may be fluorescent labels or other labels which are visualisable on tissue samples removed from patients.

Recombinant DNA technology may be used to improve the antibodies of the invention. Thus, chimeric antibodies may be constructed in order to decrease the immunogenicity thereof in diagnostic or therapeutic applications. Moreover, immunogenicity may be minimised by humanising the antibodies by CDR grafting [see European Patent 0 239 400 (Winter)] and, optionally, framework modification [EP 0 239 400; reviewed in international patent application WO 90/07861 (Protein Design Labs)].

Antibodies according to the invention may be obtained from animal serum, or, in the case of monoclonal antibodies or fragments thereof, produced in cell culture. Recombinant DNA technology may be used to produce the antibodies according to established procedure, in bacterial or preferably mammalian cell culture. The selected cell culture system preferably secretes the antibody product.

Therefore, the present invention includes a process for the production of an antibody according to the invention comprising culturing a host, e.g. E. coli or a mammalian cell, which has been transformed with a hybrid vector comprising an expression cassette comprising a promoter operably linked to a first DNA sequence encoding a signal peptide linked in the proper reading frame to a second DNA sequence encoding said protein, and isolating said protein.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. foetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

In vitro production provides relatively pure antibody preparations and allows scale-up to give large amounts of the desired antibodies. Techniques for bacterial cell, yeast or mammalian cell cultivation are known in the art and include homogeneous suspension culture, e.g. in an airlift reactor or in a continuous stirrer reactor, or immobilised or entrapped cell culture, e.g. in hollow fibres, microcapsules, on agarose microbeads or ceramic cartridges.

Large quantities of the desired antibodies can also be obtained by multiplying mammalian cells in vivo. For this purpose, hybridoma cells producing the desired antibodies are injected into histocompatible mammals to cause growth of antibody-producing tumours. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules is described in the above references and also in, for example, EP 0623679; EP 0368684 and EP 0436597, which are incorporated herein by reference.

The cell culture supernatants are screened for the desired antibodies, preferentially by immunofluorescent staining of cells expressing DNC by immunoblotting, by an enzyme immunoassay, e.g. a sandwich assay or a dot-assay, or a radioimmunoassay.

For isolation of the antibodies, the immunoglobulins in the culture supernatants or in the ascitic fluid may be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-) affinity chromatography, e.g. affinity chromatography with a DNC molecule or with Protein-A.

The invention further concerns hybridoma cells secreting the monoclonal antibodies of the invention. The preferred hybridoma cells of the invention are genetically stable, secrete monoclonal antibodies of the invention of the desired specificity and can be activated from deep-frozen cultures by thawing and recloning.

The invention also concerns a process for the preparation of a hybridoma cell line secreting monoclonal antibodies directed to DNC, characterised in that a suitable mammal, for example a Balb/c mouse, is immunised with purified DNC, an antigenic carrier containing purified DNC or with cells bearing DNC, antibody-producing cells of the immunised mammal are fused with cells of a suitable myeloma cell line, the hybrid cells obtained in the fusion are cloned, and cell clones secreting the desired antibodies are selected. For example spleen cells of Balb/c mice immunised with cells bearing DNC are fused with cells of the myeloma cell line PAI or the myeloma cell line Sp2/0-Ag14, the obtained hybrid cells are screened for secretion of the desired antibodies, and positive hybridoma cells are cloned.

Preferred is a process for the preparation of a hybridoma cell line, characterised in that Balb/c mice are immunised by injecting subcutaneously and/or intraperitoneally between 10 and 10⁷ and 10⁸ cells of human tumour origin which express DNC containing a suitable adjuvant several times, e.g. four to six times, over several months, e.g. between two and four months, and spleen cells from the immunised mice are taken two to four days after the last injection and fused with cells of the myeloma cell line PAI in the presence of a fusion promoter, preferably polyethylene glycol. Preferably the myeloma cells are fused with a three- to twentyfold excess of spleen cells from the immunised mice in a solution containing about 30% to about 50% polyethylene glycol of a molecular weight around 4000. After the fusion the cells are expanded in suitable culture media as described hereinbefore, supplemented with a selection medium, for example HAT medium, at regular intervals in order to prevent normal myeloma cells from overgrowing the desired hybridoma cells.

The invention also concerns recombinant DNAs comprising an insert coding for a heavy chain variable domain and/or for a light chain variable domain of antibodies directed to a DNC molecule as described hereinbefore. By definition such DNAs comprise coding single stranded DNAs, double stranded DNAs consisting of said coding DNAs and of complementary DNAs thereto, or these complementary (single stranded) DNAs themselves.

Furthermore, DNA encoding a heavy chain variable domain and/or for a light chain variable domain of antibodies directed to a DNC molecule can be enzymatically or chemically synthesised DNA having the authentic DNA sequence coding for a heavy chain variable domain and/or for the light chain variable domain, or a mutant thereof. A mutant of the authentic DNA is a DNA encoding a heavy chain variable domain and/or a light chain variable domain of the above-mentioned antibodies in which one or more amino acids are deleted or exchanged with one or more other amino acids. Preferably said modification(s) are outside the CDRs of the heavy chain variable domain and/or of the light chain variable domain of the antibody. Such a mutant DNA is also intended to be a silent mutant wherein one or more nucleotides are replaced by other nucleotides with the new codons coding for the same amino acid(s). Such a mutant sequence is also a degenerated sequence. Degenerated sequences are degenerated within the meaning of the genetic code in that an unlimited number of nucleotides are replaced by other nucleotides without resulting in a change of the amino acid sequence originally encoded. Such degenerated sequences may be useful due to their different restriction sites and/or frequency of particular codons which are preferred by the specific host, particularly E. coli, to obtain an optimal expression of the heavy chain murine variable domain and/or a light chain murine variable domain.

The term mutant is intended to include a DNA mutant obtained by in vitro mutagenesis of the authentic DNA according to methods known in the art.

For the assembly of complete tetrameric immunoglobulin molecules and the expression of chimeric antibodies, the recombinant DNA inserts coding for heavy and light chain variable domains are fused with the corresponding DNAs coding for heavy and light chain constant domains, then transferred into appropriate host cells, for example after incorporation into hybrid vectors.

The invention therefore also concerns recombinant DNAs comprising an insert coding for a heavy chain murine variable domain of an antibody directed DNC fused to a human constant domain g, for example γ1, γ2, γ3 or γ4, preferably γ1 or γ4. Likewise the invention concerns recombinant DNAs comprising an insert coding for a light chain murine variable domain of an antibody directed to DNC fused to a human constant domain κ or λ, preferably κ.

In another embodiment the invention pertains to recombinant DNAs coding for a recombinant polypeptide wherein the heavy chain variable domain and the light chain variable domain are linked by way of a spacer group, optionally comprising a signal sequence facilitating the processing of the antibody in the host cell and/or a DNA coding for a peptide facilitating the purification of the antibody and/or a cleavage site and/or a peptide spacer and/or an effector molecule.

The DNA coding for an effector molecule is intended to be a DNA coding for the effector molecules useful in diagnostic or therapeutic applications. Thus, effector molecules which are toxins or enzymes, especially enzymes capable of catalysing the activation of prodrugs, are particularly indicated. The DNA encoding such an effector molecule has the sequence of a naturally occurring enzyme or toxin encoding DNA, or a mutant thereof, and can be prepared by methods well known in the art.

Antibodies and antibody fragments according to the invention are useful in therapy. Accordingly, the invention provides a composition for therapy comprising an antibody according to the invention.

6. Drug Development

Many compounds according to the present invention may be lead compounds useful for drug development. Useful lead compounds are especially antibodies, and particularly intracellular antibodies expressed within the cell in a gene therapy context, which may be used as models for the development of peptide or low molecular weight therapeutics. In a preferred aspect of the invention, lead compounds and DNC may be co-crystallised in order to facilitate the design of suitable low molecular weight compounds which mimic the interaction observed with the lead compound.

Crystallisation involves the preparation of a crystallisation buffer, for example by mixing a solution of the peptide or peptide complex with a “reservoir buffer”, preferably in a 1:1 ratio, with a lower concentration of the precipitating agent necessary for crystal formation. For crystal formation, the concentration of the precipitating agent is increased, for example by addition of precipitating agent, for example by titration, or by allowing the concentration of precipitating agent to balance by diffusion between the crystallisation buffer and a reservoir buffer. Under suitable conditions such diffusion of precipitating agent occurs along the gradient of precipitating agent, for example from the reservoir buffer having a higher concentration of precipitating agent into the crystallisation buffer having a lower concentration of precipitating agent. Diffusion may be achieved for example by vapour diffusion techniques allowing diffusion in the common gas phase. Known techniques are, for example, vapour diffusion methods, such as the “hanging drop” or the “sitting drop” method. In the vapour diffusion method a drop of crystallisation buffer containing the protein is hanging above or sitting beside a much larger pool of reservoir buffer. Alternatively, the balancing of the precipitating agent can be achieved through a semipermeable membrane that separates the crystallisation buffer from the reservoir buffer and prevents dilution of the protein into the reservoir buffer.

In the crystallisation buffer the peptide or peptide/binding partner complex preferably has a concentration of up to 30 mg/ml, preferably from about 2 mg/ml to about 4 mg/ml.

Formation of crystals can be achieved under various conditions which are essentially determined by the following parameters: pH, presence of salts and additives, precipitating agent, protein concentration and temperature. The pH may range from about 4.0 to 9.0.

The concentration and type of buffer is rather unimportant, and therefore variable, e.g. in dependence with the desired pH. Suitable buffer systems include phosphate, acetate, citrate, Tris, MES and HEPES buffers. Useful salts and additives include e.g. chlorides, sulphates and other salts known to those skilled in the art. The buffer contains a precipitating agent selected from the group consisting of a water miscible organic solvent, preferably polyethylene glycol having a molecular weight of between 100 and 20000, preferentially between 4000 and 10000, or a suitable salt, such as a sulphates, particularly ammonium sulphate, a chloride, a citrate or a tartarate.

A crystal of a peptide or peptide/binding partner complex according to the invention may be chemically modified, e.g. by heavy atom derivatization. Briefly, such derivatization is achievable by soaking a crystal in a solution containing heavy metal atom salts, or a organometallic compounds, e.g. lead chloride, gold thiomalate, thimerosal or uranyl acetate, which is capable of diffusing through the crystal and binding to the surface of the protein. The location(s) of the bound heavy metal atom(s) can be determined by X-ray diffraction analysis of the soaked crystal, which information may be used e.g. to construct a three-dimensional model of the peptide.

A three-dimensional model is obtainable, for example, from a heavy atom derivative of a crystal and/or from all or part of the structural data provided by the crystallisation. Preferably building of such model involves homology modelling and/or molecular replacement.

The preliminary homology model can be created by a combination of sequence alignment with any ANC the structure of which is known, secondary structure prediction and screening of structural libraries. For example, the sequences of DNC and a candidate peptide can be aligned using suitable software.

Computational software may also be used to predict the secondary structure of the peptide or peptide complex. The peptide sequence may be incorporated into the DNC structure. Structural incoherences, e.g. structural fragments around insertions/deletions can be modelled by screening a structural library for peptides of the desired length and with a suitable conformation. For prediction of the side chain conformation, a side chain rotamer library may be employed.

The final homology model is used to solve the crystal structure of the peptide by molecular replacement using suitable computer software. The homology model is positioned according to the results of molecular replacement, and subjected to further refinement comprising molecular dynamics calculations and modelling of the inhibitor used for crystallisation into the electron density.

7. Pharmaceutical Compositions

In a preferred embodiment, there is provided a pharmaceutical composition comprising a compound or compounds identifiable by an assay method as defined in the previous aspect of the invention.

A pharmaceutical composition according to the invention may be a composition of matter comprising a compound or compounds capable of modulating the nucleoside analogue transporting activity of DNC as an active ingredient. Alternatively, the pharmaceutical compound may be a nucleoside analogue which is transported less efficiently than other therapeutically useful nucleoside analogues, and thus has a reduced toxicity.

The active ingredients of a pharmaceutical composition comprising the active ingredient according to the invention are contemplated to exhibit excellent therapeutic activity, for example, in the treatment of tumours or viral diseases, when administered in amount which depends on the particular case. Dosage regima may be adjusted to provide the optimum therapeutic response. For example, several divided doses may be administered daily or the dose may be proportionally reduced as indicated by the exigencies of the therapeutic situation.

The active ingredient may be administered in a convenient manner such as by the oral, intravenous (where water soluble), intramuscular, subcutaneous, intranasal, intradermal or suppository routes or implanting (e.g. using slow release molecules). Depending on the route of administration, the active ingredient may be required to be coated in a material to protect said ingredients from the action of enzymes, acids and other natural conditions which may inactivate said ingredient.

In order to administer the active ingredient by other than parenteral administration, it will be coated by, or administered with, a material to prevent its inactivation. For example, the active ingredient may be administered in an adjuvant, co-administered with enzyme inhibitors or in liposomes. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin.

Liposomes include water-in-oil-in-water CGF emulsions as well as conventional liposomes.

The active ingredient may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof, and vegetable oils. The proper fluidity can be maintained, for example, by the use of a coating such as lecithin, by the maintenance of the required particle size in the case of dispersion and by the use of superfactants.

The prevention of the action of microorganisms can be brought about by various antibacterial and antifungal agents, for example, parabens, chlorobutanol, phenol, sorbic acid, thirmerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active ingredient in the required amount in the appropriate solvent with various of the other ingredients enumerated above, as required, followed by filtered sterilisation. Generally, dispersions are prepared by incorporating the sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredient is suitably protected as described above, it may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active ingredient may be incorporated with excipients and used in the form of ingestible tablets, buccal tablets, troches, capsules, elixirs, suspensions, syrups, wafers, and the like. The amount of active ingredient in such therapeutically useful compositions in such that a suitable dosage will be obtained.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active ingredient, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active ingredient may be incorporated into sustained-release preparations and formulations.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such as active material for the treatment of disease in living subjects having a diseased condition in which bodily health is impaired.

The principal active ingredients are compounded for convenient and effective administration in effective amounts with a suitable pharmaceutically acceptable carrier in dosage unit form. In the case of compositions containing supplementary active ingredients, the dosages are determined by reference to the usual dose and manner of administration of the said ingredients.

In a further aspect there is provided the active ingredient of the invention as hereinbefore defined for use in the treatment of disease. Consequently there is provided the use of an active ingredient of the invention for the manufacture of a medicament for the treatment of disease associated with nucleoside analogue toxicity.

Moreover, there is provided a method for treating a condition associated with nucleoside analogue toxicity, comprising administering to a subject a therapeutically effective amount of a compound or compounds identifiable using an assay method as described above.

The invention is further described, for the purpose of illustration only, in the following examples.

### EXAMPLES

### General Methods

cDNA Sequencing

To extend the EST sequence in 5′- and 3′-directions, PCRs were performed on adaptor-ligated double-stranded human liver cDNA (1 ng, CLONTECH; ref. 8) with primers AP1 and AP2 (CLONTECH) (NotI sites replaced by BamHI). Products were cloned into the pUC19 vector. The sequences of inserts were determined and assembled (8).

Bacterial Expression and Protein Purification

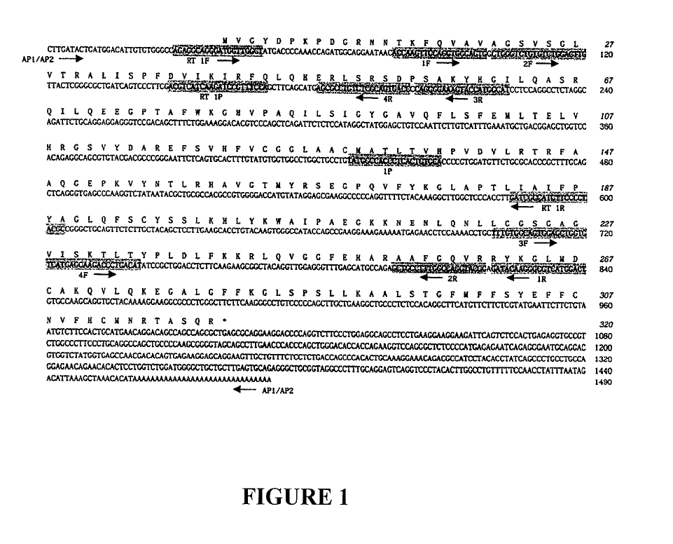

The coding sequence was amplified from human cDNA by PCR with nucleotides 39-58 and 981-998 of the cDNA sequence (FIG. 1) as primers. The product was cloned into the pET21b vector. Transformants of Escherichia coli DH5a were selected on ampicillin (100 mg/ml) and screened by colony PCR and restriction digestion of plasmids. The sequences of inserts were verified. The encoded protein had additional C-terminal leucine and glutamate residues, followed by six histidines. The protein was overexpressed in E. coli BL21(DE3) (9). Purified inclusion bodies (9), suspended in buffer [10 mM NaCl/20 mM Pipes (pH 8.0)] were solubilized in sarkosyl [1.67% (wt/vol)] for 5 min at 0° C. This solution was diluted 20 times with buffer [0.1% sarkosyl/0.5 M NaCl/20 mM Pipes (pH 8.0)] and centrifuged (12,000×g, 10 min, 4° C.). The supernatant was put onto a Ni⁺-NTA-agarose column (Qiagen, Chatsworth, Calif.). Impurities were removed by decreasing the pH of the buffer (0.1% sarkosyl/20 mM Pipes) from 8.0 to 6.5. DNC eluted in the same buffer at pH 6.2. Proteins were analysed by SDS/PAGE and stained with Coomassie blue. Their N termini were sequenced (8). The amount of pure DNC was estimated by laser densitometry of stained samples (8).

Transport Assays

The recombinant protein in sarkosyl was reconstituted into liposomes in the presence of substrates (10). Both cardiolipin (1.14 mg/ml) and EDTA (1 mM) were added. Transport was measured at 25° C. with internal and external pHs at 6.8. It was started by adding [α-35S]dATP or [¹⁴C]-ADP and terminated after 2 min by addition of 100 mM of p-chloromercuribenzene sulfonate (10). Entrapped radioactivity was counted (10). The initial transport rate was calculated from the radioactivity taken up by proteoliposomes within 2 min (in the initial linear range). Other transport activities were assayed similarly (10). The amount of DNC incorporated into liposomes was measured as described (11) and varied between 15 and 20% of the protein added to the reconstitution mixture.

Expression Analysis

Total human and mouse RNAs (2 mg) from various tissues were reverse transcribed with random hexamers or an oligo(dT)16 primer (final volume of 40 ml). Half of the reaction product was used as PCR template with forward and reverse primers RT 1F and RT 1R, respectively (FIG. 1), to amplify a cDNA fragment. The products were probed with radiolabeled oligonucleotide RT 1P (FIG. 1). As a control, a 384-bp cDNA fragment of β-actin was amplified from the rest of the reaction product with the primers 5′-GTTTGAGACCTTCAA-CACCC-3′ and 5′-CCAATGGTGATGACCTGGCC-3′. Mitochondria from rat tissues were solubilized in SDS. Proteins were separated by SDS/PAGE, transferred to nitrocellulose (11), and exposed to a rabbit antiserum against human DNC. Immuno-conjugates were detected with a secondary antibody (horseradish peroxidase coupled to anti-rabbit Ig) and 3,3′-diaminobenzidine as peroxidase substrate.

### Example 1

Sequence of the Human DNC

By phylogenetic analysis of the sequences of all C. elegans mitochondrial carriers and of mammalian carriers of known function, a seven-protein subfamily related to the ANC was found. With their sequences, a human EST (THC91779) that encoded a related sequence was identified. THC91779 overlapped three other human EST clones (THC90932, THC132863, and N40412). This partial cDNA of 769 nucleotides was extended to the final sequence (FIG. 1), which encodes a protein with a molecular mass of 34,588. The assignment of the translational initiation codon is consistent with an inframe stop codon 27 base pairs upstream. The protein sequence has the characteristics of the family of mitochondrial carriers. One C. elegans clone (C42C1.10) is 39% identical to the human sequence over residues 1-364 of its 649-amino acid sequence.

Characterisation of Recombinant DNC

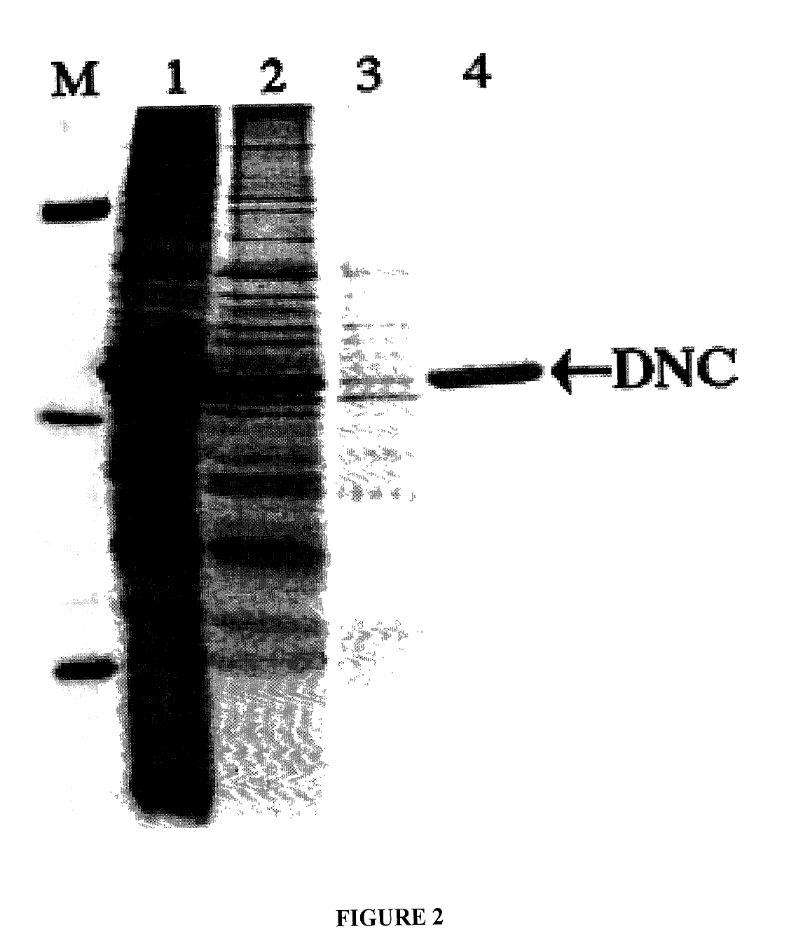

The DNC accumulated as inclusion bodies in E. coli BL21(DE3) (see FIG. 2, lane 1). The purified protein was homogeneous (FIG. 2, lane 4) with an apparent molecular mass of 36 kDa (calculated value with initiator methionine and His-tail, 36,310). Its N-terminal sequence (VGYDPKPDGR) is identical to residues 2-11 of the protein encoded in the cDNA. About 80 mg of purified protein was obtained per litre of culture.

### Example 2

Transport Properties of DNC

The reconstituted human DNC catalysed the exchange of [α-35 S]dATP for dADP or ADP with first-order kinetics (rate constant 0.02 min⁻¹), isotopic equilibrium being approached exponentially (see FIG. 3a). Uptake of external substrate required internal substrate. It did not catalyse homo-exchanges of malate, fumarate, oxoglutarate, carnitine, glutamate, aspartate, glutamine, or ornithine (internal concentration, 10 mM; external concentration, 1 mM). Transport under either saturating or nonsaturating concentrations of external [α-35 S]dATP (1 mM and 0.02 mM, respectively) with 10 mM internal ADP, had a sharp pH optimum at 6.8. The recombinant DNC had its highest affinity for ADP and dNDPs from the internal side of the proteoliposomal membrane (FIG. 3b). The highest rates of [α-35 S]dATP uptake into proteoliposomes were with internal ADP or dADP. High activities also were found with the other internal dNDPs; significant activities were found with GDP, CDP, and UDP; much lower activities with NTPs, dNTPs, dNMPs, and pyrophosphate; and virtually no activity with NMPs and NADH, adenine, deoxyadenosine, phosphate, oxoglutarate, citrate, glycine, carnitine, adenosine, guanosine, cytidine, uridine, deoxyguanosine, deoxycytidine, deoxythymidine, deoxyuridine, guanine, cytosine, thymine, or uracil. The exchange of pyrophosphate, but not of adenine or deoxyadenosine, shows that phosphate groups are essential for transport, but as pyrophosphate exchange was 22% of ADP exchange, the nucleoside moiety is also important. Dideoxynucleoside triphosphates also exchanged with dATP at twice the rate of dNTPs (see FIG. 3b).

External nucleoside and deoxynucleoside mono-, di-, and triphosphates inhibited the [α-35 S]dATP/ADP exchange (see FIG. 3c). Nucleoside diphosphates were more effective than either triphosphates or monophosphates. Deoxynucleotides were more potent than the corresponding nucleotides, and dideoxynucleoside triphosphates were as effective as dNDPs. The rate of dATP uptake was more sensitive to purine than pyrimidine nucleotides, and adenine nucleotides inhibited better than guanine nucleotides. Adenosine, guanosine, cytidine, uridine, deoxyadenosine, deoxyguanosine, deoxycytidine, deoxythymidine, deoxyuridine, adenine, guanine, cytosine, thymine, and uracil had no effect.

The uptake of 50 mM [α-35 S]dATP (internal substrate, 10 mM ADP; reaction time, 2 min) was inhibited completely by 0.1 mM p-chloromercuribenzene sulfonate and 10 mM pyridoxal 5′-phosphate (inhibitors of many mitochondrial carriers), and partly (41%) by 10 mM bathophenathroline [another strong inhibitor of several mitochondrial carriers (5-9)]. High concentrations of carboxyatractyloside (0.1 mM) and bongkrekate (0.01 mM) (inhibitors of the ANC) were partly effective on the DNC (42 and 37% inhibition, respectively). A specific inhibitor of the mitochondrial citrate carrier, 2 mM 1,2,3-benzenetricarboxylate, reduced the dATP/ADP exchange rate to 40%, possibly because this compound as the optimal substrate for DNC carries three negative charges. No significant inhibition was observed with 2 mM butylmalonate, phenylsuccinate, a-cyano-4-hydroxycinnamate and N-ethylmaleimide (inhibitors of other characterised mitochondrial carriers), and 0.033 mM cytochalasin B (inhibitor of a plasma membrane nucleoside transporter).

The exchange rate of internal ADP or dADP (10 mM) depended on the external concentration of [α-35 S]dATP (20-1,000 mM) or [¹⁴C]ADP (8-400 mM). With both external substrates, linear functions were obtained in double-reciprocal plots. They were independent of the internal substrate and intersected the ordinate close to a common point. For ADP and DATP, the transport affinities (Km) were 42.6±4.7 μM and 106±15 μM (mean values of 6 and 63 experiments, respectively). The average value of Vmax was 0.85±0.15 μmol/min per gram of protein. Several external substrates were competitive inhibitors of [α-35 S]DATP uptake (Table 1). They increased the apparent Km without change in Vmax. These results confirm that DADP is the highest-affinity external substrate (Ki 14 μM). Furthermore, the Ki values of all of the dNDPs are two to three times and four to five times lower than those of their corresponding NDPs or dNTPs, respectively. The affinity of the DNC for ddNTPs is very high (Ki for ddATP, 25 μM) and similar to that of the dNDPs. The Ki of the antiviral drug ddCTP is 70 μM.

Tissue Distribution

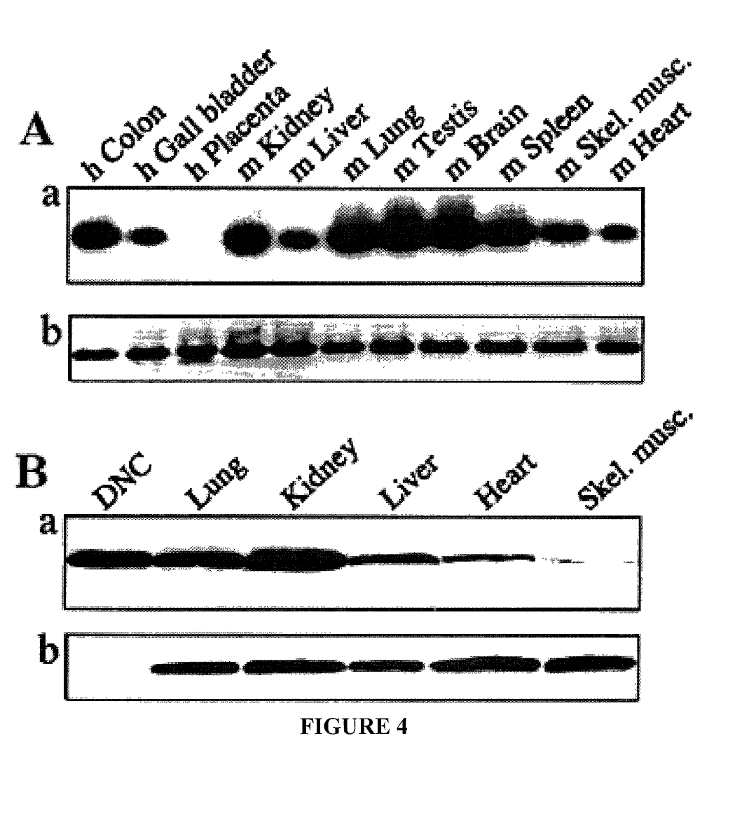

High levels of mRNA for the DNC were detected in colon, kidney, lung, testis, spleen, and brain, and lower amounts in gall bladder, liver, skeletal muscle, and heart (FIG. 4A). Similarly abundant levels of protein expression were found in rat mitochondria from kidney, lung, and liver, and lower levels in skeletal muscle and heart (FIG. 4B). The only tissue with no detectable DNC transcripts was human placenta, possibly because RNA was extracted postpartum, when biosynthetic activity is low.

### References

1. Walker, J. E. (1992) Curr. Opin. Struct. Biol. 2, 519-526.

2. Palmieri, F. (1994) FEBSLett. 346, 48-54.

3. Palmieri, F. & van Ommen, B. (1999) in Frontiers in Cellular Bioenergetics, eds. Papa, S., Guerrieri, F. & Tager, J. M. (Kluwer AcademicyPlenum, New York), pp. 489-519.

4. Palmieri, L., De Marco, V., Iacobazzi, V., Palmieri, F., Runswick, M. J. & Walker, J. E. (1997) FEBSLett. 410, 447-451.

5. Palmieri, L., Lasorsa, F. M., De Palma, A., Palmieri, F., Runswick, M. J. & Walker, J. E. (1997) FEBSLett. 417, 114-118.

6. Palmieri, L., Vozza, A., Agrimi, G., De Marco, V., Runswick, M. J., Palmieri, F. & Walker, J. E. (1999) J. Biol. Chem. 274, 22184-22190.

7. Palmieri, L., Agrimi, G., Runswick, M. J., Fearnley, I. M., Palmieri, F. & Walker, J. E. (2001) J. Biol. Chem. 276, 1916-1922.

8. Fiermonte, G., Palmieri, L., Dolce, V., Lasorsa, F. M., Palmieri, F., Runswick, M. J. & Walker, J. E. (1998) J. Biol. Chem. 273, 24754-24759.

9. Fiermonte, G., Walker, J. E. & Palmieri, F. (1993) Biochem. J. 294, 293-299.

10. Palmieri, F., Indiveri, C., Bisaccia, F. & lacobazzi, V. (1995) Methods Enzymol. 260, 349-369.

11. Fiermonte, G., Dolce, V. & Palmieri F. (1998) J. Biol. Chem. 273, 22782-22787.

12. Muller, V., Basset, G., Nelson, D. R. & Klingenberg, M. (1996) Biochemistry 35, 16132-16143.

13. Schwabe, J. W., Chapman, L., Finch, J. T. &Rhodes, D. (1993) Cell 75, 567-578.

14. Gonzalez-Barroso, M. M., Fleury, C., Jimenez, M. A., Sanz, J. M., Romero, A., Bouillaud, F. & Rial, E. (1999) J. Mol. Biol. 292, 137-149.

15. Engstrom, Y. & Rozell, B. (1988) EMBO J. 7, 1615-1620.

16. Parsons, P. & Simpson, M. V. (1973) J. Biol. Chem. 248, 1912-1919.

17. Chang, C. N., Skalski, V., Zhou, J. H. & Cheng, Y. C. (1992) J. Biol. Chem. 267, 22414-22420.

18. Enriquez, J. A., Ramos, J., Perez-Martos, A., Lopez-Perez, M. J. & Montoya, J. (1994) Nucleic Acids Res. 22, 1861-1865.

19. Letko, G., Kuster, U., Duszynski, J. & Kunz, W. (1980) Biochim. Biophys. Acta 593, 196-203.

20. Zhu, C., Johansson, M., Permert, J. &Karlsson, A. (1998) Biochem. Pharmacol. 56, 1035-1040.

21. De Clercq, E. (1991) J. Acquired Immune Defic. Syndr. 4, 207-218.

22. Pedrol, E., Masanes, F., Femandez-Sola, J., Cofan, M., Casademont, J., Grau, J. M. & Urbano-Marquez, A. (1996) J. Neurol. Sci. 138, 42-48.

23. Lewis, W. & Dalakas, M. C. (1995) Nat. Med. 1, 417-422.

24. Agarwal, R. P. & Olivero, O. A. (1997) Mutat. Res. 390, 223-231.

25. Benbrik, E., Chariot, P., Bonavaud, S., Ammi-Said, M., Frisdal, E., Rey, C., Gherardi, R. & Barlovatz-Meimon, G. (1997) J. Neurol. Sci. 149, 19-25.

26. Brinkman, K., ter Hofstede, H. J. M., Burger, D. M., Smeitink, J. A. & Koopmans, P. P. (1998) AIDS12, 1735-1744.

27. Dalakas, M. C., Illa, I., Pezeshkpour, G., Laukaitis, J., Cohen, B. & Griffin, J. (1990) N. Engl. J Med. 322, 1098-1105.

28. Lewis, W., Simpson, J. F. & Meyer, R. R. (1994) Circ. Res. 74, 344-348.

29. Bridges, E. G., Abdesslem, F. &Sommadossi, J. -P. (1993) Biochem. Pharmacol. 45,1571-1576.

All publications mentioned in the present specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A mitochondrial deoxynucleotide carrier (DNC) which transports deoxynucleoside diphosphates, wherein said carrier:
a) catalyses the exchange of DATP for DADP or ADP with first-order kinetics and a rate constant of about 0.02 min⁻¹;
b) has a pH optimum at about pH 6.8; and
c) exchanges dATP more efficiently from dNDPs than for NTPs, dNTP, dNMPs and pyrophosphate.

2. A mitochondrial DNC according to claim 1 which has a calculated molecular mass of about 34,588.

3. A mitochondrial DNC according to claim 1 which is a mammalian DNC.

4. A mitochondrial DNC according to claim 3, which is a human DNC.

5. A mitochondrial deoxynucleotide carrier (DNC) which transports deoxynucleoside diphosphates, wherein said carrier:
a) has the amino acid sequence set forth in SEQ. ID. No. 2; or
b) has an amino acid sequence as set forth in SEQ. ID. No. 2, including one or more amino acid additions, deletions or substitutions, and retains the ability to transport deoxynucleoside diphosphates; or
c) is encoded by a nucleic acid sequence set forth in SEQ. ID. No. 1.

6. A nucleic acid encoding a polypeptide according to claim 1 or 5.

7. A nucleic acid according to claim 6, which comprises a nucleotide sequence selected from the group consisting of: the nucleotide sequence of: (a) SEQ. ID. No. 1; (b) the coding portion of the nucleotide sequence SEQ. ID. No. 1; and (c) a nucleotide sequence which is at least 80% homologous to (a) or (b); and (d) a nucleotide sequence at least 20 nucleotides in length which is selectively hybridisable with (a), (b) or (c) or the complement thereof.

8. A nucleic acid according to claim 6 which is labeled.

9. A method for selecting a nucleoside analogue, comprising assaying the efficiency with which the nucleoside analogue is transported by a DNC according to claim 1 or 5; and selecting those analogues which are least effectively transported.

10. A method according to claim 9, comprising the steps of:
a) incubating a DNC according to claim 1 or 5 with a nucleoside analogue in a transport modeling system;
b) assessing the efficiency of transport of the nucleoside analogue;
c) repeating steps a) and b) with one or more further nucleoside analogues; and
d) comparing the efficiency of transport for the tested nucleoside analogues.

11. A method according to claim 10 wherein a reference efficiency of transport is determined for a nucleoside analogue, and further nucleoside analogues are compared against the reference value.

12. A method for identifying a compound or compounds capable, directly or indirectly, of modulating the transport of nucleoside analogues by a DNC according to claim 1 or 5, and thereby the toxicity of said nucleoside analogues, comprising the steps of:
(a) incubating a DNC according to the invention with the compound or compounds to be assessed; and
(b) identifying those compounds which influence the activity of the DNC.

13. A method for identifying a modulator of nucleoside analogue-induced mitochondrial toxicity, comprising the steps of:
(a) incubating a DNC molecule with the compound or compounds to be assessed; and
(b) identifying those compounds which bind to the DNC molecule.

14. A method according to claim 13, conducted in the presence of one or more nucleoside analogues.

15. A method according to claim 13 which further comprises the step of: (c) assessing the compounds which bind to DNC for the ability to modulate DNC activity in a transport assay.

16. A method according to any one of claims 11, or 13 wherein the DNC is incubated in a transport modelling system.

17. A method according to claim 16, wherein the transports modelling system measures the efficiency of transport of nucleoside analogue across a membrane.
